(19) United States
(12) Patent Application Publication
(10) Pub. No.: US 2014/0105845 A1
(43) Pub. Date: Apr. 17, 2014

(54) COSMETIC COMPOSITIONS COMPRISING WAX DISPERSIONS

(71) Applicant: L'OREAL, Paris (FR)

(72) Inventors: Hy Si Bui, Piscataway, NJ (US); Christopher Pang, New York, NY (US); Jean-Thierry Simonnet, Mamaroneck, NY (US); Bradford Pistorio, Clark, NJ (US)

(73) Assignee: L'OREAL, PARIS (FR)

(21) Appl. No.: 13/652,295

(22) Filed: Oct. 15, 2012

(51) Int. Cl. A61K 8/92; A61Q 1/10

(52) U.S. Cl. 424/70.7

(57) ABSTRACT

The disclosure relates to compositions comprising at least one film former and at least one wax dispersion. The disclosure further relates to methods of improving the ease of removability of compositions.

### TECHNICAL FIELD

The present disclosure relates to compositions comprising film forming agents and wax dispersions. The present disclosure also relates to methods of improving the ease of removability of compositions.

### BACKGROUND

Consumer products, such as cosmetics and personal care products, may employ ingredients that allow these products to form a film or coating on various substrates, such as keratinous substrates (e.g., hair and skin). Inclusion of a film former in a cosmetic composition can improve various properties, such as, for example, shine, adhesion, and long wear.

In particular, the use of latex film formers in cosmetic and personal care products is known, such as, for example, in mascara compositions, hair styling products, topical foundations, sunscreen compositions, and water-based nail enamels. For example, U.S. patent application Ser. No. 13/341,875 discloses latex film formers for use in nail compositions. Latex and latex blends have been used to provide extended-wear properties of the product into which they are formulated. For example, conventional washable mascara compositions may use latex film formers in combination with an oil-in-water emulsion. Further, extended-wear mascara compositions comprising latex film forming agents are known, such as, for example, in U.S. Provisional Patent Application Nos. 61/541,173; 61/542,131; and 61/542,136, all of which are incorporated herein in their entireties.

However, cosmetic and personal care products that incorporate latex film formers, such as extended-wear mascara compositions, typically demonstrate difficulty in removability from the keratinous substrates to which they are applied, which is at least in part due to the inclusion of the latex film former. Thus, there is a need in the industry to provide extended wear cosmetic and personal care products which are more easily removed by the consumer.

It has now been surprisingly discovered that the incorporation of a wax dispersion into a composition comprising a latex film former can improve the ease of removability of the composition from keratinous substrates.

### SUMMARY

The disclosure relates to compositions, such as cosmetic compositions (e.g. mascara compositions, such as extended-wear mascara compositions) and/or personal care products, said compositions comprising:
a. at least one film former, such as a latex film former; and
b. a wax dispersion comprising:
   i. solid wax particles having a particle size ranging from equal to or greater than about 1 micron up to about 60 microns;
   ii. a surfactant mixture comprising:
      1. at least one nonionic surfactant; and
      2. at least one ionic surfactant; and
   iii. water.

Furthermore, the disclosure also relates to methods of improving the ease of removability of compositions, such as cosmetic compositions (e.g. mascara compositions, such as extended-wear mascara compositions) and/or personal care products, said methods comprising incorporating a wax dispersion according to various embodiments of the disclosure into said compositions.

### BRIEF DESCRIPTION OF THE FIGURES

FIG. 1 shows optical microscopy views of wax dispersions comprising solid particles of beeswax at 10 microns, 20 microns, and 80 microns; and

FIG. 2 represents the difference in ease of removability among compositions comprising film former and 0% wax dispersion, 5% wax dispersion, or 10% wax dispersion, having a particle size of 10 microns, 20 microns, or 80 microns.

### DESCRIPTION OF EXEMPLARY EMBODIMENTS

The disclosure relates, in various exemplary embodiments, to compositions, e.g. cosmetic compositions and/or personal care products, comprising at least one film former and at least one wax dispersion. In various exemplary embodiments, the composition is a mascara composition, such as an extended-wear mascara composition. In at least certain exemplary embodiments, the cosmetic compositions are aqueous.

In various exemplary embodiments, the cosmetic composition comprising the at least one film former and at least one wax dispersion exhibits an increased ease of removability relative to similar compositions that do not comprise the at least one wax dispersion, although it should be noted that in certain exemplary embodiments, a greater or lesser degree of increased ease of removability may be exhibited, and in further embodiments, increased ease of removability may not be exhibited.

In at least one exemplary embodiment, the cosmetic compositions are chosen from aqueous, extended-wear compositions, e.g. extended-wear mascara, comprising at least one film former and at least one wax dispersion. By “extended-wear,” it is meant compositions that typically do not need reapplication during the day, once a consumer applies the composition initially, such as, for example, over a period of time of about 24 hours or more, such as from about 12-16 hours. In certain embodiments, the aqueous, extended-wear mascara compositions may be water-proof and/or smudge-proof, although in at least certain embodiments, the mascara composition may not be one or both of water-proof and/or smudge-proof.

In at least certain embodiments according to the disclosure, the composition comprising at least one film former and at least one wax dispersion exhibits reduced or minimized stickiness or tackiness that is otherwise generally attributed to the use of waxes in cosmetic compositions, such as, for example, as found in traditional mascara compositions comprising emulsions of water and traditional waxes. However, in certain embodiments, reduced or minimized stickiness or tackiness may not be observed.

Film Formers

As used herein, the terms “film former,” “film-forming agent,” “film-forming polymer,” and variations thereof, mean an agent or polymer capable, by itself or in the presence of an auxiliary film-forming agent, of forming a substantially continuous film that adheres to a support, and especially to keratin materials. Among the film-forming agents that may be used, mention may be made of, for example, latex film formers.

Exemplary and non-limiting latex film formers useful in various embodiments may be chosen from, for example, acrylate copolymers, styrene/acrylate copolymers, acrylamide/acrylate copolymers, polyurethanes, derivatives thereof, and mixtures thereof.

In accordance with various exemplary embodiments, acrylate copolymers may be chosen from copolymers comprising two or more monomers chosen from acrylic acid, methacrylic acid, and their simple esters, for example, lower alkyl esters such as methyl, ethyl, and ethylhexyl esters. By way of non-limiting example only, acrylate copolymers may be chosen from ammonium acrylates copolymers, ethyl acrylates copolymers, acrylates/ethylhexylacrylate copolymers, acrylates/octylacrylates copolymers, akly(meth)acrylates copolymers, acrylates/C₁₂-C₂₂ alkylmethacrylate copolymers, ethylacrylate/methacrylic acid copolymer, and t-butyl acrylate/ethyl acrylate/methacrylic acid copolymer. Exemplary commercial acrylate copolymers include, but are not limited to, ALLIANZ™ OPT sold by Ashland Specialty Ingredients; COVACRYL A15 and COVACRYL E14 sold by Sensient Cosmetic Technologies LCW; DAITOSOL 4000 SJT, DAITOSOL 5000 AD, DAITOSOL 5000 SJ, KOBOGUARD® 50A, and KOBOGUARD® 50N sold by Kobo Products, Inc.; DERMACRYL® AQF, YODOSOL 32A707, YODOSOL GH15, YODOSOL GH32, YODOSOL GH33, YODOSOL GH34, YODOSOL GH35, YODOSOL GH800, and YODOSOL GH810 sold by AkzoNobel; LUVIFLEX® SOFT, LUVIMER® 36D, and LUVIMER® 100P sold by BASF; and NEOCRYL XK-90 sold by Neoresins, Inc.

According to further exemplary embodiments of the disclosure, styrene/acrylate copolymers may be chosen from styrene/acrylates/ammonium methacrylate copolymers, such as SYNTRAN® 5760 sold by Interpolymer Corporation. Other non-limiting examples of commercially available styrene/acrylate copolymers include, but are not limited to, DAITOSOL 5000 STY sold by Kobo Products, Inc.; JONCRYL® 77 sold by BASF; NEOCRYL BT-62 sold by Neoresins, Inc.; RHOPLEX™ P-376 and UCAR™ DL 432S sold by Dow Chemical Company; and YODOSOL GH41 and YODOSOL GH840 sold by AkzoNobel.

In further exemplary embodiments, acrylamide/acrylate copolymers may be chosen from acrylic acid/ethyl acrylate/t-butyl acrylamide copolymer, acrylates/octylacrylamide copolymer, and octylacrylamide/acrylates/methacrylates copolymer. Exemplary commercial acrylamide/acrylate copolymers include, but are not limited to AMPHOMER® LV-71 and DERMACRYL® 79 sold by AkzoNobel and ULTRAHOLD® STRONG sold by BASF.

According to additional exemplary embodiments, polyurethanes may be chosen from polyurethane-32, polyurethane-34, and polyurethane-35. Exemplary commercial polyurethanes include, but are not limited to, products sold by Bayer under the trade name BAYCUSAN®, such as BAYCUSAN® C1000, BAYCUSAN® C1001, BAYCUSAN® C1003, and BAYCUSAN® C1004.

In at least one exemplary embodiment, the latex film former may be chosen from blends comprising a combination of latex film formers, including, for example, a blend of any of the above-mentioned film formers. By way of non-limiting example, a latex film forming blend useful according to the disclosure may comprise (1) at least one random styrene acrylate copolymer or derivatives thereof, and at least one acrylate copolymer or derivatives thereof, or (2) at least two random styrene acrylate copolymers or derivatives thereof.

According to at least certain exemplary embodiments of the disclosure, the at least one latex film former may be chosen from those having a glass transition temperature (Tg) ranging from about −15° C. to about 90° C., such as from about 0° C. to about 50° C.

In accordance with various exemplary embodiments, the at least one latex film former may be present in the cosmetic composition in an amount, or a combined amount when more than one latex film former is used, ranging up to about 90% by weight, such as up to about 70%, up to about 50%, up to about 40%, up to about 30%, up to about 20%, up to about 15%, or up to about 10%. In various exemplary embodiments, the at least one latex film former is present in the composition in an amount of from about 5% to about 50%, about 10% to about 30%, or about 15% to about 20%, such as about 15%, about 16%, about 17%, about 18%, about 19%, or about 20%.

In addition to at least one latex film former, the compositions may, in at least certain exemplary embodiments, optionally further comprise at least one coalescent and/or plasticizer. It is known that inclusion of a coalescent agent promotes the coalescence of polymer particles in an aqueous dispersion, and inclusion of a plasticizer makes it possible to plasticize a polymer in an aqueous dispersion. Any coalescent and/or plasticizer may be used, and one of skill in the art will be able to choose an appropriate coalescent and/or plasticizer with little or no routine experimentation based on, for example, the type of cosmetic composition being formulated and the desired properties thereof.

Optional coalescents and/or plasticizers useful according to various exemplary embodiments of the disclosure include, but are not limited to, those disclosed in U.S. Pat. No. 6,372,201. By way of example only, optional plasticizers may be chosen from tributyl citrate, texanol ester alcohol, diisobutyl adipate, the ester of tertbutyl acid and 2,2,4-trimethylpentane-1,3-diol, diethyl adipate, diethyl phthalate, dibutyl phthalate, dioctyl phthalate, butyl 2-ethylhexyl phthalate, dimethyl sebacate, dibutyl sebacate, ethyl stearate, 2-ethylhexyl palmitate, dipropylene glycol n-butyl ether, and mixtures thereof. By way of example only, optional coalescents may be chosen from propylene glycol n-butyl ether, dipropylene glycol dibenzoate, dipropylene glycol dimethyl ether, propylene glycol methyl ether acetate, propylene glycol propyl ether, methyl lactate, ethyl lactate, isopropyl lactate, and mixtures thereof.

Wax Dispersion

Exemplary wax dispersions useful according to various embodiments of the disclosure include, but are not limited to, aqueous wax dispersions comprising solid particles of at least one wax, a surfactant, and water.

In various exemplary embodiments, the wax dispersion useful according to the present disclosure can be prepared with a surfactant mixture comprising a combination of nonionic and ionic surfactants, and following an emulsification process. In at least certain exemplary embodiments, an amphoteric surfactant may optionally also be used.

The wax dispersion according to various embodiments of the present disclosure may be employed in an amount ranging up to about 90% by weight, such as up to about 80% by weight, up to about 60% by weight, up to about 40% by weight, up to about 30% by weight, up to about 25% by weight, up to about 20% by weight, up to about 15% by weight, up to about 10% by weight, or up to about 5% by weight, based on the total weight of the composition according to the present disclosure, including all ranges and subranges therebetween. In at least one embodiment, the wax dispersion may be present in the composition in an amount of about 10%.

Wax

The amount of solid wax particles present in the wax dispersion according to various embodiments may range from about 10-60%, such as about 15-50%, about 20-45%, or about 30-40%, by weight, relative to the weight of the wax dispersion. For example, the solid wax particles may be present in an amount of about 10%, about 15%, about 20%, about 25%, about 30%, about 35%, about 40%, about 45%, or about 50%, by weight, based on the total weight of the wax dispersion, including all ranges and subranges therebetween.

In at least certain embodiments, the wax dispersion may be employed in a composition such that the amount of the at least one wax comprising the solid wax particles of the wax dispersion ranges from about 1% to about 20% by weight, such as from about 1.5% to about 10% by weight, about 2% to about 8% by weight, or from about 2% to about 5% by weight, relative to the total weight of the composition.

The solid wax particles of the present disclosure may be chosen from particles of natural and synthetic waxes. Natural waxes may include, for example, one or a combination of animal, vegetable/plant, mineral, or petroleum derived waxes. They are typically esters of fatty acids and long chain alcohols. Wax esters are derived from a variety of carboxylic acids and a variety of fatty alcohols. The waxes comprising the solid wax particle of the present disclosure may also be known as solid lipids.

Examples of waxes comprising the at least one solid wax particle of the present disclosure include, but are not limited to, beeswax, hydrogenated alkyl olive esters (commercially available under the trade name phytowax olive), carnauba wax, candelilla wax, ouricoury wax, Japan wax, cork fibre wax or sugar cane wax, rice wax, montan wax, paraffin wax, lignite wax or microcrystalline wax, ceresin or ozokerite, palm kernel glycerides/hydrogenated palm glycerides and hydrogenated oils such as hydrogenated castor oil or jojoba oil, sugarcane, retamo, bayberry, rice bran, soy, castor, esparto, japan waxes, hydroxyoctacosanyl hydroxystearate, Chinese wax, cetyl palmitate, lanolin, shellac, and spermaceti; synthetic waxes such as those of the hydrocarbon type and polyethylene waxes obtained from the polymerization or copolymerization of ethylene, and Fischer-Tropsch® waxes, or else esters of fatty acids, such as octacosanyl stearate, glycerides which are solid at temperatures of above 35° C., silicone waxes, such as alkyl- or alkoxydimethicones having an alkyl or alkoxy chain ranging from 10 to 45 carbon atoms, poly(di)methylsiloxane esters which are solid at 30° C. and whose ester chain comprising at least 10 carbon atoms, or else di(1,1,1-trimethylolpropane)tetrastearate, which is sold or manufactured by Heterene under the name HEST® 2T-4S, and mixtures thereof.

Other examples of waxes or solid lipids include C20-40 di- and triglycerides, including those which contain unsaturated fatty acids, C20-40 fatty alcohols, C2-40 fatty amines and their compounds, and sterols. Yet further examples include Hest 2T-5E-4S, Ditrimethylolpropane tetralaurate, Koster BK-34, Fluoro Polymethylalkyl dimethylsiloxane, Blend of Dilauryl Adipate and Ditetradecyl Adipate, Astrocaryum MuruMuru Seed Butter, Myrica Pubescens Wax, PEG-70 Mango Glycerides, oxypropylenated lanolin wax, and hydrogenated Coco-glycerides.

By way of non-limiting example only, the wax may, in various embodiments, advantageously be chosen from beeswax, commercially available from various suppliers, hydrogenated stearyl olive ester, and commercially available from the supplier Sophim under the tradename, Phytowax Olive 18 L 57, hydrogenated myristyl olive ester, and commercially available from the supplier Sophim under the tradename, Phytowax Olive 14 L 48, VP/eicosene copolymer, commercially available from the supplier ISP under the tradenames, Antaron® V 220 or Ganex® V 220F, and ditrimethyloylpropane tetrastearate, commercially available from the supplier Heterene under the tradename, HEST 2T-4S.

Other suitable waxes include silsesquioxane resin waxes such as C30-45 alkyldimethylsilyl propylsilsesquioxane, commercially available as DOW CORNING SW-8005 C30 Resin Wax, from the company Dow Corning and such as those described in WO2005/100444.

The wax(es) which comprises the at least one solid wax particle of the present disclosure may be chosen from soft waxes and from hard waxes. Soft waxes may be defined as those waxes which have a melting point of below about 70° C., and preferably, a melting point of below about 60° C. Hard waxes may be defined as those waxes which have a melting point of equal to or greater than about 70° C., and preferably, a melting point of equal to or greater than about 60° C.

According to one embodiment, soft waxes according to the present disclosure include, but are not limited to, Paraffin wax, stearic alcohol, ozokerite, synthetic beeswax, beeswax, candelilla wax, PVP/eicosene copolymer, hydrogenated jojoba wax, palm butter, sumac wax, polyglyceryl beeswax, tricontanyl/PVP, siliconyl beeswax, stearyl stearate, ceresin wax, hydrogenated myristyl olive esters (e.g., phytowax olive 14 L 48), hydrogenated stearyl olive esters (e.g., phytowax olive 18 L 57), Koster K82P, orange peel wax, Pentaerythritol distearate, Theobroma Grandiflorum Seed Butter, silicone resin wax, Polymethylalkyl dimethylsiloxane, Pentaerythrityl tetrastearate, Tetracontanyl Stearate, fatty acid wax, behenyl alcohol, alkyl dimethicone wax, Stearyl Benzoate, Berry wax, koster wax, siliconyl candelilla wax, Ditrimethylolpropane tetrastearate, Clariant Licowax KST 1, Dipentaerythrytol hexastearate, Ditrimethylolpropane tetrabehenate, Behenyl methacrylate gréffé PDMS, jojoba esters, waxolive, inholive, phytowax ricin 16 L 64, hydrogenated macadamia seed oil, synthetic wax, dooctadecyl carbonate, montan wax, lemon peel extract, ditrimethyloylpropane tetrastearate, and C30-45 alkyldimethylsilyl propylsilsesquioxane. (check melting points of last two)

According to one embodiment, hard waxes according to the present disclosure, include, but are not limited to, carnauba wax, microcrystalline wax, polyethylene wax, hydrogenated castor oil, wax AC 540, Hydroxyoctacosanyl Hydroxystearate, hydrogenated castor wax, wax AC 400, rice bran wax, C20-40 alkyl stearate, Alcohol polyethylene wax, octanedioate, sunflower seed wax, fischer-tropsch wax, Chinese insect wax, shellac wax, benehyl fumarate, synthetic wax, betsawax RX-13750, phytowax ricin 22 L 73, and vegetable wax.

The solid wax particles of the wax dispersions may, in various embodiments, comprise a particle size ranging from equal to or greater than about 1 micron up to about 80 microns, such as up to about 60 microns, up to about 50 microns, up to about 25 microns, up to about 20 microns, up to about 17.5 microns, up to about 15 microns, up to about 12.5 microns, up to about 10 microns, up to about 7.5 microns, up to about 5 microns, or up to about 2.5 microns, including all ranges and subranges therebetween. In at least one exemplary embodiment, the solid wax particles of the wax dispersion comprise a particle size of about 20 microns. In yet a further exemplary embodiment, the solid wax particles of the dispersion comprise a particle size of about 15 microns. In yet a further exemplary embodiment, the solid wax particles of the dispersion comprise a particle size of about 10 microns. In yet a further exemplary embodiment, the solid wax particles of the dispersion comprise a particle size of about 7.5 microns.

The term “particle size” as used herein refers to the diameter of the solid wax particle, and is meant to indicate an average particle diameter of all solid wax particles in the dispersion. For non-spherical particles, the particle size refers to the largest diameter of the particles, i.e., the diameter in the dimension having the largest diameter.

The shape of the solid wax particles in various exemplary wax dispersions may be any shape, such as, for example, spherical or ellipsoidal or oval. The terms “spherical” or “ellipsoidal” or “oval” as used herein also mean that the solid wax particle has a uniform and substantially spherical or ellipsoidal or oval shape. The term “substantially” as used in the context of the shape of a spherical particle means that the particle is of substantially isotropic shape, i.e., it has a relatively regular morphology. It should be noted, however, that a line of symmetry is not required when the solid wax particle has a spherical shape.

Further, the solid wax particles of the wax dispersions may optionally have surface texturing, such as lines or indentations or protuberances that are relatively small in scale when compared to the overall size of the solid wax particle, and still be considered by those of skill in the art to be substantially spherical or ellipsoidal or oval.

The solid wax particles in the wax dispersions according to the present disclosure may, in at least certain embodiments, be substantially homogeneous with respect to their shape and/or particle size distribution, although it is not required. The term “substantially” as used in this context means that about 50% or more of the solid wax particles in an aqueous dispersion of the present disclosure are of the same spherical, ellipsoidal, or oval shape and of the same particle size distribution.

The particle size and shape of the solid wax particle of the present disclosure may be evaluated by any known method, such as those described in US patent application number 2006/0292095, for example, laser diffraction, ultrasonic extinction (acoustic spectroscopy), photo cross-correlation spectroscopy, and image analysis (microscopy).

Surfactant Mixture

The surfactant mixture of the present disclosure comprises, in at least certain exemplary embodiments, at least one nonionic surfactant and at least one ionic surfactant. In further exemplary embodiments, the surfactant mixture may also comprise at least one amphoteric surfactant.

In general, nonionic surfactants having a Hydrophilic-Lipophilic Balance (HLB) of from at least 5, such as from about 5 to about 20, or such as from about 5 to about 15, can be chosen in various exemplary embodiments. HLB is understood to mean the balance between the size and strength of the hydrophilic group and the size and strength of the lipophilic group of the surfactant. Nonlimiting examples of nonionic surfactants are disclosed in McCutcheon's “Detergents and Emulsifiers,” North American Edition (1986), published by Allured Publishing Corporation; and McCutcheon's “Functional Materials,” North American Edition (1992); both of which are incorporated by reference herein in their entirety.

Examples of nonionic surfactants useful herein include, but are not limited to, alkoxylated derivatives of the following: fatty alcohols, alkyl phenols, fatty acids, fatty acid esters and fatty acid amides, wherein the alkyl chain is in the C₁₂-C₅₀ range, such as in the C₁₆-C₄₀ range, or in the C₂₄-C₄₀ range, and having from about 1 to about 110 alkoxy groups. The alkoxy groups are chosen from C₂-C₆ oxides and their mixtures, with ethylene oxide, propylene oxide, and their mixtures being chosen in certain embodiments. The alkyl chain may be linear, branched, saturated, or unsaturated. Of these alkoxylated non-ionic surfactants, the alkoxylated alcohols are chosen in at least certain embodiments, such as ethoxylated alcohols and propoxylated alcohols. The alkoxylated alcohols may be used alone or in mixtures thereof. The alkoxylated alcohols may also be used in mixtures with those alkoxylated materials disclosed herein-above.

Other representative examples of such ethoxylated fatty alcohols include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10) steareth-10 (a stearyl alcohol ethoxylate having an average degree of ethoxylation of 10), and steareth-2 (a stearyl alcohol ethoxylate having an average degree of ethoxylation of 2), steareth-100 (a stearyl alcohol ethoxylate having an average degree of ethoxylation of 100), beheneth-5 (a behenyl alcohol ethoxylate having an average degree of ethoxylation of 5), beheneth-10 (a behenyl alcohol ethoxylate having an average degree of ethoxylation of 10), and other derivatives and mixtures of the preceding.

Also available commercially are Brij® nonionic surfactants from Uniqema, Wilmington, Del. Typically, Brij® is the condensation products of aliphatic alcohols with from about 1 to about 54 moles of ethylene oxide, the alkyl chain of the alcohol being typically a linear chain and having from about 8 to about 22 carbon atoms, for example, Brij® 72 (i.e., Steareth-2) and Brij® 76 (i.e., Steareth-10).

Also useful herein as nonionic surfactants are alkyl glycosides, which are the condensation products of long chain alcohols, e.g. C₈-C₃₀ alcohols, with sugar or starch polymers. These compounds can be represented by the formula (S)n-O—R wherein S is a sugar moiety such as glucose, fructose, mannose, galactose, and the like; n is an integer of from about 1 to about 1000, and R is a C₈-C₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Examples of these surfactants are alkyl polyglucosides wherein S is a glucose moiety, R is a C₈-C₂₀ alkyl group, and n is an integer of from about 1 to about 9. Commercially available examples of these surfactants include decyl polyglucoside (available as APG® 325 CS) and lauryl polyglucoside (available as APG® 600CS and 625 CS), all the above-identified polyglucosides APG® are available from Cognis, Ambler, Pa. Also useful herein are sucrose ester surfactants such as sucrose cocoate and sucrose laurate.

Other nonionic surfactants suitable for use include glyceryl esters and polyglyceryl esters and their derivatives, including but not limited to, glyceryl monoesters, such as glyceryl monoesters of C₁₆-C₂₂ saturated, unsaturated and branched chain fatty acids such as glyceryl oleate, glyceryl monostearate, glyceryl monoisostearate, glyceryl monopalmitate, glyceryl monobehenate, and mixtures thereof, and polyglyceryl esters of C₁₆-C₂₂ saturated, unsaturated and branched chain fatty acids, such as polyglyceryl-4 isostearate, polyglyceryl-3 oleate, polyglyceryl-2 sesquioleate, triglyceryl diisostearate, diglyceryl monooleate, tetraglyceryl monooleate, and mixtures thereof. glyceryl ester derivatives include, but are not limited to, polyethylene glycol ethers of glyceryl esters such as PEG-30 glyceryl stearate, PEG-30 glyceryl diisostearate, PEG-30 glyceryl isostearate, PEG-30 glyceryl laurate, PEG-30 glyceryl oleate, and mixtures thereof.

Also useful herein as nonionic surfactants are sorbitan esters. For example, sorbitan esters of C₁₆-C₂₂ saturated, unsaturated and branched chain fatty acids, may be chosen. Because of the manner in which they are typically manufactured, these sorbitan esters usually comprise mixtures of mono-, di-, tri-, etc. esters. Representative examples of suitable sorbitan esters include sorbitan monooleate (e.g., SPAN® 80), sorbitan sesquioleate (e.g., Arlacel® 83 from Uniqema, Wilmington, Del.), sorbitan monoisostearate (e.g., CRILL® 6 from Croda, Inc., Edison, N.J.), sorbitan stearates (e.g., SPAN® 60), sorbitan trioleate (e.g., SPAN® 85), sorbitan tristearate (e.g., SPAN® 65), sorbitan palmitate (e.g., SPAN® 40), and sorbitan isostearate. Sorbitan palimtate and/or sorbitan sesquioleate may be chosen in at least certain exemplary embodiments.

Also suitable for use herein are alkoxylated derivatives of glyceryl esters, sorbitan esters, and alkyl polyglycosides, wherein the alkoxy groups is chosen from C₂-C₆ oxides and their mixtures, with ethoxylated or propoxylated derivatives of these materials being chosen in certain particular embodiments. Nonlimiting examples of commercially available ethoxylated materials include TWEEN® (ethoxylated sorbitan mono-, di- and/or tri-esters of C₁₂ to C₁₈ fatty acids with an average degree of ethoxylation of from about 2 to about 20).

In certain embodiments, nonionic surfactants may be chosen from those formed from a fatty alcohol, a fatty acid, or a glyceride with a C₄ to C₃₆ carbon chain, e.g. a C₁₂ to C₁₈ carbon chain or a C₁₆ to C₁₈ carbon chain, derivatized to yield an HLB of at least 8. Such derivatives can be polymers such as ethoxylates, propoxylates, polyglucosides, polyglycerins, polylactates, polyglycolates, polysorbates, and others that would be apparent to one of ordinary skill in the art. Such derivatives may also be mixed polymers of the above, such as ethoxylate/propoxylate species, where the total HLB is optionally greater than or equal to 8. For example, the nonionic surfactants contain ethoxylate in a molar content of from 10-25, such as from 10-20 moles.

In certain exemplary embodiments, the nonionic surfactants of the present disclosure are chosen from polyethylene glycol ethers of glyceryl esters, PEG-30 glyceryl stearate and sorbitan esters such as sorbitan palmitate. In at least one embodiment, the at least one nonionic surfactant is chosen from polysiloxane emulsifying polymers, including, by way of example only, ABIL CARE 85, ABIL EM 90, and ABIL EM 97.

The nonionic surfactant will typically be employed in an amount of from about 60% to about 95% by weight, or from about 65% to about 90% by weight, or from about 70% to about 90% by weight, based on the total weight of the surfactant mixture of the present disclosure.

Typically, the ionic surfactants contain a lipophilic hydrocarbon group and a polar functional hydrophilic group.

The following anionic surfactants, which may be used alone or as mixtures, may be mentioned: mention may be made especially of the salts, in particular the alkali metal salts such as the sodium salts, the ammonium salts, the amine salts, the amino alcohol salts or the salts of alkaline-earth metals, for example of magnesium, of the following compounds: alkyl sulfates, alkyl ether sulfates, alkylamido ether sulfates, alkylaryl polyether sulfates, monoglyceride sulfates; alkylsulfonates, alkyl phosphates, alkylamidesulfonates, alkylarylsulfonates, α-olefin sulfonates, paraffin sulfonates; alkyl sulfosuccinates, alkyl ether sulfosuccinates, alkylamide sulfosuccinates; alkyl sulfoacetates; acylsarcosinates; and acylglutamates, the alkyl or acyl groups of all these compounds comprising from 6 to 24 carbon atoms and the aryl group, such as denoting a phenyl or benzyl group. It is also possible to use esters of C6-C24 alkyl and of polyglycoside-carboxylic acids, such as alkyl glucoside citrates, polyalkyl glycoside tartrates and polyalkyl glycoside sulfosuccinates; alkyl sulfosuccinamates, acyl isethionates and N-acyltaurates, the alkyl or acyl group of all these compounds containing from 12 to 20 carbon atoms. Among the anionic surfactants that may also be used, mention may also be made of acyl lactylates in which the acyl group contains from 8 to 20 carbon atoms. Mention may also be made of alkyl-D-galactosideuronic acids and salts thereof, and also polyoxyalkylenated (C6-C24)alkylether-carboxylic acids, polyoxyalkylenated (C6-C24)alkyl(C6-C24)arylethercarboxylic acids and polyoxyalkylenated (C6-C24)alkylamidoethercarboxylic acids and salts thereof, in particular those comprising from 2 to 50 ethylene oxide groups, and mixtures thereof.

Among the anionic surfactants which may be chosen, mention may also be made of the salts, in particular of sodium, of magnesium or of ammonium, of alkyl sulfates; of alkyl ether sulfates, for instance sodium lauryl ether sulfate, e.g. containing 2 or 3 mol of ethylene oxide; of acyl glutamates, for instance, disodium stearoyl glutamate and sodium stearoyl glutamate; of alkyl ether carboxylates; and mixtures thereof, the alkyl or acyl groups generally containing from 6 to 24 carbon atoms, such as from 8 to 16 carbon atoms.

Among the cationic surfactants, mention may be made of:

i) alkylpyridinium salts, ammonium salts of imidazoline, diquaternary ammonium salts, and ammonium salts containing at least one ester function;

ii) quaternary ammonium salts having the following general formula:

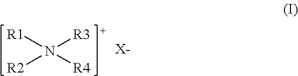

in which the radicals R1 to R4, which may be identical or different, represent a linear or branched aliphatic radical containing from 1 to 30 carbon atoms, or an aromatic radical such as aryl or alkylaryl; the aliphatic radicals may optionally comprise heteroatoms (O, N, S or halogens) and may optionally, be substituted.

The aliphatic radicals are chosen, for example, from C12-C22 alkyl, alkoxy, C2-C6 polyoxyalkylene, alkylamide, (C12-C22)alkylamido(C2-C6)alkyl, (C12-C22)alkyl-acetate and hydroxyalkyl radicals, containing from 1 to 30 carbon atoms. X— is an anion chosen halides, phosphates, acetates, lactates, C2-C6 alkyl sulfates and alkyl or alkylarylsulfonates.

iii) quaternary ammonium salts of imidazoline of formula:

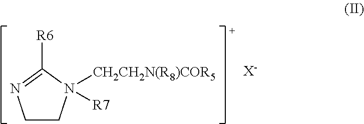

in which:

R5 represents an alkenyl or alkyl radical containing from 8 to 30 carbon atoms, for example fatty acid derivatives of tallow or of coconut,

R6 represents a hydrogen atom, a C1-C4 alkyl radical or an alkenyl or alkyl radical containing from 8 to 30 carbon atoms,

R7 represents a C1-C4 alkyl radical,

R8 represents a hydrogen atom or a C1-C4 alkyl radical,

X′ is an anion chosen from halides, phosphates, acetates, lactates, C2-C6 alkyl sulfates, alkylsulfonates or alkylarylsulfonates.

R5 and R6 may denote a mixture of alkenyl or alkyl radicals containing from 12 to 21 carbon atoms, such as, for example, fatty acid derivatives of tallow, R7 denotes methyl and R8 denotes hydrogen. Such a product is, for example, Quaternium-27 (CTFA 1997) or Quaternium-83 (CTFA 1997), which are sold under the names Rewoquat® W75, W90, W75PG and W75HPG by the company Witco,

iv) diquaternary ammonium salts of formula:

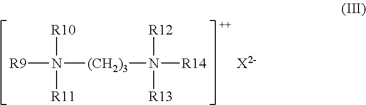

in which:

R9 denotes an aliphatic radical containing from about 16 to 30 carbon atoms,

R10, R11, R12, R13 and R14, which may be identical or different, are chosen from hydrogen and an alkyl radical containing from 1 to 4 carbon atoms, and

X— is an anion chosen from halides, acetates, phosphates, nitrates, ethyl sulfates and methyl sulfates.

Such diquaternary ammonium salts in particular comprise propanetallowediammonium dichloride;

v) quaternary ammonium salts containing at least one ester function, such as those of formula:

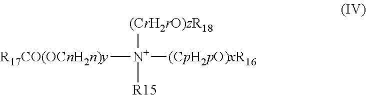

in which:

R15 is chosen from C1-C6 alkyl radicals and C1-C6 hydroxyalkyl or dihydroxyalkyl radicals;

R16 is chosen from the radical R19-CO—, linear or branched, saturated or unsaturated C1-C22 hydrocarbon-based radicals R20, a hydrogen atom;

R18 is chosen from the radical R21-CO, linear or branched, saturated or unsaturated C1-C22 hydrocarbon-based radicals R22, a hydrogen atom;

R17, R19 and R21, which may be identical or different, are chosen from linear or branched, saturated or unsaturated C7-C21 hydrocarbon-based radicals;

r, n and p, which may be identical or different, are integers ranging from 2 to 6;

y is an integer ranging from 1 to 10;

x and z, which may be identical or different, are integers ranging from 0 to 10;

X— is a simple or complex organic or mineral anion;

with the proviso that when the sum x+y+z is from 1 to 15, that when x is 0, then R16 denotes R20 and that when z is 0, then R18 denotes R22.

The alkyl radicals R15 may be linear or branched, and more particularly linear. Optionally, R15 denotes a methyl, ethyl, hydroxyethyl or dihydroxypropyl radical, and more particularly a methyl or ethyl radical.

In certain exemplary embodiments, the sum x+y+z may range from 1 to 10.

When R16 is a hydrocarbon-based radical R20, it may contain from 12 to 22 carbon atoms, or contain from 1 to 3 carbon atoms.

When R18 is a hydrocarbon-based radical R22, it may contain 1 to 3 carbon atoms.

Optionally, R17, R19 and R21, which may be identical or different, are chosen from linear or branched, saturated or unsaturated C11-C21 hydrocarbon-based radicals, and more particularly from linear or branched, saturated or unsaturated C11-C21 alkyl and alkenyl radicals.

Optionally, x and z, which may be identical or different, are equal to 0 or 1. In at least one embodiment, y is equal to 1.

Optionally, r, n and p, which may be identical or different, are equal to 2 or 3 and even more particularly equal to 2.

The anion X— may be a halide (chloride, bromide or iodide) or a C1-C4 alkyl sulfate, such as methyl sulfate. The anion X— may also represent methanesulfonate, phosphate, nitrate, tosylate, an anion derived from an organic acid (such as acetate or lactate), or any other anion that is compatible with the ammonium containing an ester function.

The surfactants may be, for example, the salts (chloride or methyl sulfate) of diacyloxyethyldimethylammonium, of diacyloxyethylhydroxyethyl-dimethylammonium, of monoacyloxyethylhydroxyethyldimethylammonium, of triacyloxy-ethylmethylammonium, of monoacyloxyethylhydroxyethyldimethylammonium, and mixtures thereof. The acyl radicals may contain 14 to 18 carbon atoms and are more particularly derived from a plant oil, for instance palm oil or sunflower oil. When the compound contains several acyl radicals, these radicals may be identical or different. Such compounds are sold, for example, under the names Dehyquart® by the company Cognis, Stepanquat® by the company Stepan, Noxamium® by the company Ceca, and Rewoquat®WE 18 by the company Rewo-Goldschmidt.

vi) quaternary ammonium salts and in particular behenyltrimethylammonium chloride, dipalmitoylethylhydroxyethylmethylammonium methosulfate, cetyltrimethylammonium chloride, quaternium-83, behenylamidopropyl-2,3-di hydroxypropyldimethylammonium chloride and palmitylamidopropyltrimethyl-ammonium chloride.

Other suitable cationic surfactants are esterquats which are quaternary ammonium compounds having fatty acid chains containing ester linkages.

Among the useful cationic surfactants, mention may be made of compounds of formula (I) chosen from cetrimonium chloride, behentrimonium chloride, Behenyl PG-Trimonium chloride, dicetyl dimonium chloride, and mixtures, thereof.

Other optional cationic surfactant are esterquats chosen from Dibehenoylethyl Dimonium Chloride, Dipalmitoylethyl Dimonium Chloride, Distearoylethyl Dimonium Chloride, Ditallowoyl PG-dimonium Chloride, Dipalmitoylethyl hydroxyethylmonium methosulfate, Distearoylethyl hydroxyethylmonium methosulfate, and mixtures, thereof.

Without wishing to be bound by theory, it is believed that the presence of an ionic surfactant, particularly, at the time of making the dispersion, reduces or minimizes the aggregation of the solid wax particles in the aqueous dispersion of the present disclosure. Thus, the surfactant mixture comprising at least one ionic surfactant may act as a dispersant to facilitate the uniform dispersion of the solid wax particles and to enhance the stabilization of the dispersion itself.

In certain embodiments of the present disclosure, the surfactant mixture contains at least one nonionic surfactant and at least one ionic surfactant comprising at least one anionic surfactant.

In other embodiments, the surfactant mixture contains at least one nonionic surfactant and at least one ionic surfactant comprising at least one cationic surfactant.

In at least certain exemplary embodiments, the surfactant mixture contains at least one nonionic surfactant and at least one ionic surfactant comprising at least one anionic surfactant, wherein the surfactant mixture is free of cationic surfactants.

In yet other exemplary embodiments, the surfactant mixture contains at least one nonionic surfactant and at least one ionic surfactant comprising at least one cationic surfactant, wherein the surfactant mixture is free of anionic surfactants.

The at least one ionic surfactant may be employed in an amount of from about 5% to about 40% by weight, or from about 5% to 30% by weight, or from about 5% to about 20% by weight, based on the total weight of the surfactant mixture of the present disclosure.

In certain embodiments, the surfactant mixture, that is, the combined amount of the at least one nonionic surfactant and the at least one ionic surfactant, is present in the aqueous dispersion in an amount of from about 1.0% to about 5% by weight, such as from about 1.5% to about 3.5% by weight, or from about 1.5% to about 3% by weight, based on the total weight of the aqueous dispersion.

In certain exemplary embodiments, the surfactant mixture of the present disclosure is free of amphoteric surfactants. However, in other exemplary embodiments, at least one amphoteric surfactant may be added.

Amphoteric surfactants include, but are not limited to, aliphatic secondary or tertiary amine derivatives, in which the aliphatic group is a linear or branched chain containing 8 to 22 carbon atoms and containing at least one water-soluble anionic group, such as, for example, a carboxylate, sulfonate, sulfate, phosphate or phosphonate group; mention may also be made of (C8-C20)alkylbetaines, sulfobetaines, (C8-C20)alkyl-amido-(C6-C8)-alkyl-betaines or (C8-C20)alkyl-amido-(C6-C8)-alkylsulfobetaines; and mixtures thereof.

Among the amine derivatives that may be mentioned are amphocarboxyglycinate compounds and amphocarboxypropionate compounds, in particular, disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

Among the amphoteric surfactants that may be used are (C8-C20)alkylbetaines, (C8-C20)alkylamido(C6-C8)alkylbetaines and alkylamphodiacetates, and mixtures thereof.

Additional Components

The wax dispersion may optionally further comprise additional components such as solvents (including water), oils, emulsifying polymers, sunscreen agents, pigments/dyes/colorants, silicas, talc, clays, perfumes, and any other component typically found in cosmetic or personal care compositions, such as mascara compositions, or dispersions intended for use in such compositions. Such additional ingredients may optionally be added during the time of making the wax dispersion in order to either improve/modify the physical properties of the solid wax particles and/or to allow the solid wax particles to provide other benefits in addition to the benefits obtained from waxes.

Suitable oils include, but are not limited to, mineral oils (paraffin); plant oils and natural oils (sweet almond oil, macadamia oil, grapeseed oil, olive oil, argan oil, tocopherol or vitamin E, shea butter oil, jojoba oil); synthetic oils, for instance perhydrosqualene, fatty acids or fatty esters (for instance the C₁₂-C₁₅ alkyl benzoate sold under the trade name Finsolv® TN, commercially available from Innospec or Tegosoft® TN, commercially available from Evonik Goldschmidt, octyl palmitate, isopropyl lanolate and triglycerides, including capric/caprylic acid triglycerides), oxyethylenated or oxypropylenated fatty esters and ethers; or fluoro oils, and polyalkylenes.

Other oils include for example: silicone oils, for instance volatile or non-volatile polymethylsiloxanes (PDMS) with a linear or cyclic silicone chain, which are liquid or pasty at room temperature, especially cyclopolydimethylsiloxanes (cyclomethicones) such as cyclohexasiloxane; polydimethyl-siloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenyl-siloxanes, diphenyl dimethicones, diphenylmethyl-diphenyltrisiloxanes or 2-phenylethyl trimethylsiloxy silicates, and polymethylphenylsiloxanes; mixtures thereof.

Other suitable oils include, but are not limited to, volatile hydrocarbon-based oils such as, for example, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, isohexadecane, and for example, the oils sold under the trade names of Isopar™ or Permethyl®, and their mixtures.

Other suitable oils include esters such as those of formula R₁COOR₂ in which R₁ represents a linear or branched higher fatty acid residue containing from 1 to 40 carbon atoms, including from 7 to 19 carbon atoms, and R₂ represents a branched hydrocarbon-based chain containing from 1 to 40 carbon atoms, including from 3 to 20 carbon atoms, and also including, for example, octyldodecyl neopentanoate, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate, and pentaerythritol esters. Other suitable esters include polyesters, alkoxylated esters, and alkoxylated polyesters. Vitamin E oil may also be chosen.

The oils may also be chosen from silicones. Suitable silicones include, but are not limited to, the silicone oils described above and other silicones such as non-volatile silicones such as dimethicone fluids having viscosity values of equal to or greater than 300 cst, and pentaphenyldimethicone, also known as trimethyl pentaphenyl trisiloxane, commercially available from Dow Corning under the tradename Dow Corning® 555.

The wax dispersion according to various embodiments of the present disclosure may also comprise an emulsifying polymer, e.g. an amphiphilic polymer.

Among the emulsifying polymers that are suitable for use according to the disclosure, mention may be made of:

POE-POP diblock and triblock copolymers such as those described in U.S. Pat. No. 6,464,990;

polyoxyethylenated silicone surfactants such as those described in U.S. Pat. No. 6,120,778;

non-crosslinked hydrophobic AMPSs such as those described in EP 1 466 588;

amphiphilic acrylic polymers, such as PEMULEN TR-1 or TR-2 or equivalent;

the associative and gelling polymers described in US 2003/0138465;

heat-gelling polymers such as those described in patent applications US 2004/0214913, US 2003/0147832 and US 2002/0198328 and FR2856 923.

When they are present, the emulsifying polymer(s) may be introduced in a content ranging from 0.1 percent to 15 percent by weight, or even from 0.1 percent to 10 percent by weight and more particularly from 0.1 percent to 5 percent by weight relative to the total weight of the aqueous dispersion.

The wax dispersion according to various embodiments of the present disclosure may further comprise one or more sunscreen agents. Representative sunscreen agents may be chosen from organic and inorganic sunscreens or UV filters.

The organic sunscreen agents are chosen from water-soluble organic screening agents, fat-soluble organic screening agents or agents which are insoluble in the solvents presently included in suntan products, and mixtures thereof.

The organic sunscreen agents are especially chosen from cinnamic derivatives; anthranilates; salicylic derivatives; dibenzoylmethane derivatives; camphor derivatives; benzophenone derivatives; beta, beta-diphenylacrylate derivatives; triazine derivatives; benzotriazole derivatives; benzalmalonate derivatives; benzimidazole derivatives; imidazolines; bis-benzazolyl derivatives; p-aminobenzoic acid (PABA) derivatives; methylenebis(hydroxyphenylbenzotriazole) derivatives; benzoxazole derivatives; screening polymers and screening silicones; alpha-alkylstyrene-derived dimers; 4,4-diarylbutadienes; merocyanin derivatives; and mixtures thereof.

Examples of complementary organic photoprotective agents include those denoted hereinbelow under their INCI name:

Cinnamic Derivatives: Ethylhexyl Methoxycinnamate marketed in particular under the trademark “Parsol MCX®” by DSM Nutritional Products, Inc., Isopropyl Methoxycinnamate, Isoamyl p-Methoxycinnamate marketed under the trademark “Neo Heliopan E 1000®” by Symrise, DEA Methoxycinnamate, Diisopropyl Methylcinnamate, Glyceryl Ethylhexanoate Dimethoxycinnamate.

Dibenzoylmethane Derivatives: [Butyl Methoxydibenzoyl methane marketed especially under the trademark “Parsol 1789®” by DSM Nutritional Products, Inc., Isopropyl Dibenzoylmethane.

Para-Aminobenzoic Acid Derivatives: PABA, Ethyl PABA, Ethyl Dihydroxypropyl PABA, Ethylhexyl Dimethyl PABA marketed in particular under the trademark “Escalol 507®” by ISP, Glyceryl PABA, PEG-25 PABA marketed under the trademark “Uvinul P25®” by BASF.

Salicylic Derivatives: Homosalate marketed under the trademark “Eusolex HMS®” by Merck KGaA/EMD Chemicals, Inc. and EMD Chemicals Inc, Ethylhexyl Salicylate marketed under the trademark “Neo Heliopan OS®” by Symrise, Dipropylene Glycol Salicylate marketed under the trademark “Dipsal™” by Lubrizol Advanced Materials, Inc., TEA Salicylate marketed under the trademark “Neo Heliopan® TS” by Symrise.

Diphenylacrylate Derivatives: Octocrylene marketed in particular under the trademark “Uvinul N539T®” by BASF, Etocrylene marketed in particular under the trademark “Uvinul® N35” by BASF.

Benzophenone Derivatives: Benzophenone-1 marketed under the trademark “Uvinul® 400” by BASF, Benzophenone-2 marketed under the trademark “Uvinul® D50” by BASF, Benzophenone-3 or Oxybenzone marketed under the trademark “Uvinul® M40” by BASF, Benzophenone-4 marketed under the trademark “Uvinul® MS40” by BASF, Benzophenone-5, Benzophenone-6 marketed under the trademark “Helisorb® 11” by Norquay, Benzophenone-8, Benzophenone-9, Benzophenone-12, n-Hexyl 2-(4-diethylamino-2-hydroxybenzoyl)benzoate marketed under the trademark “Uvinul® A+” by BASF.

Benzylidenecamphor Derivatives: 3-Benzylidenecamphor manufactured under the trademark “Mexoryl™ SD” by Chimex, 4-Methylbenzylidenecamphor marketed under the trademark “Eusolex® 6300” by Merck, Benzylidene Camphor Sulfonic acid manufactured under the trademark “Mexoryl™ SL” by Chimex, Camphor Benzalkonium Methosulfate manufactured under the trademark “Mexoryl™ SO” by Chimex, Terephthalylidene Dicamphor Sulfonic acid manufactured under the trademark “Mexoryl™ SX” by Chimex, Polyacrylamidomethyl Benzylidene Camphor manufactured under the trademark “Mexoryl™ SW” by Chimex.

Phenylbenzimidazole Derivatives: Phenylbenzimidazole Sulfonic acid marketed in particular under the trademark “Eusolex® 232” by Merck and EMD INC., Disodium Phenyl Dibenzimidazole Tetrasulfonate marketed under the trademark “Neo Heliopan® AP” by Symrise.

Phenylbenzotriazole Derivatives: Drometrizole Trisiloxane, Methylene bis(Benzotriazolyl)Tetramethylbutylphenol, or in micronized form as an aqueous dispersion under the trademark “Tinosorb® M” by BASF.

Triazine Derivatives: bis-Ethylhexyloxyphenol Methoxyphenyl Triazine marketed under the trademark “Tinosorb® S” by BASF, Ethylhexyl Triazone marketed in particular under the trademark “Uvinul® T150” by BASF, Diethylhexyl Butamido Triazone marketed under the trademark “Uvasorb® HEB” by 3V Group, 2,4,6-Tris(dineopentyl 4′-aminobenzalmalonate)-s-triazine, 2,4,6-Tris(diisobutyl 4′-aminobenzalmalonate)-s-triazine, 2,4-Bis(n-butyl 4′-aminobenzoate)-6-(aminopropyltri-siloxane)-s-triazine, 2,4-Bis(dineopentyl 4′-aminobenzalmalonate)-6-(n-butyl 4′-amino-benzoate)-s-triazine, triazine agents, especially 2,4,6-tris(biphenyl-1,3,5-triazines (in particular 2,4,6-tris(biphenyl-4-yl)-1,3,5-triazine and 2,4,6-tris(terphenyl)-1,3,5-triazine.

Anthranilic Derivatives: Menthyl anthranilate marketed under the trademark “Neo Heliopan® MA” by Symrise.

Imidazoline Derivatives: Ethyl hexyl Dimethoxybenzylidene Dioxoimidazoline Propionate.

Benzalmalonate Derivatives: Polyorganosiloxane containing benzalmalonate functions, for instance Polysilicone-15, marketed under the trademark “Parsol® SLX” by DSM Nutritional Products, Inc.

4,4-Diarylbutadiene Derivatives: 1,1-Dicarboxy(2,2′-dimethylpropyl)-4,4-diphenylbutadiene.

Benzoxazole derivatives: 2,4-Bis[5-(1-dimethylpropyl)benzoxazol-2-yl(4-phenyl)imino]-6-(2-et-hylhexyl)imino-1,3,5-triazine marketed under the trademark Uvasorb K 2A by Sigma 3V, and mixtures thereof.

Optional organic sunscreen agents may be chosen from among: Ethylhexyl Methoxycinnamate, Ethylhexyl Salicylate, Homosalate, Butyl Methoxydibenzoylmethane, Octocrylene, Phenylbenzimidazole Sulfonic Acid, Benzophenone-3, Benzophenone-4, Benzophenone-5, n-Hexyl 2-(4-diethylamino-2-hydroxybenzoyl)benzoate, 4-Methylbenzylidene camphor, Terephthalylidene Dicamphor Sulfonic Acid, Disodium Phenyl Dibenzimidazole Tetrasulfonate, Methylene bis-Benzotriazolyl Tetramethylbutylphenol, Bis-Ethylhexyloxyphenol Methoxyphenyl Triazine, Ethylhexyl Triazone, Diethylhexyl Butamido Triazone, 2,4,6-Tris(dineopentyl 4′-aminobenzalmalonate)-s-triazine, 2,4,6-Tris(diisobutyl 4′-aminobenzalmalonate)-s-triazine, 2,4-Bis(n-butyl 4′-aminobenzoate)-6-(aminopropyltrisiloxane)-s-triazine, 2,4-Bis(dineopentyl 4′-aminobenzalmalonate)-6-(n-butyl 4′-aminobenzoate)-s-triazine, 2,4,6-Tris(biphenyl-4-yl)-1,3,5-triazine, 2,4,6-Tris(terphenyl)-1,3,5-triazine, Drometrizole Trisiloxane, Polysilicone-15, 1,1-Dicarboxy(2,2′-dimethylpropyl)-4,4-diphenylbutadiene, 2,4-Bis[5-1-(dimethylpropyl)benzoxazol-2-yl(4-phenyl)imino]-6-(2-et-hylhexyl)-imino-1,3,5-triazine, and mixtures thereof.

Examples of inorganic sunscreen agents or UV filters include, but are not limited to, metal oxide pigments which may be chosen from zinc oxide, titanium oxide, iron oxide, zirconium oxide, cerium oxide, and mixtures thereof.

The metal oxide pigments may be coated or uncoated.

The coated pigments are pigments that have undergone one or more surface treatments of chemical, electronic, mechanochemical and/or mechanical nature with compounds such as amino acids, beeswax, fatty acids, fatty alcohols, anionic surfactants, lecithins, sodium potassium, zinc, iron or aluminum salts of fatty acids, metal alkoxides (of titanium or of aluminum), polyethylene, silicones, proteins (collagen, elastin), alkanolamines, silicon oxides, metal oxides or sodium hexametaphosphate.

In certain embodiments, the sunscreen agents that may be comprise the solid wax particle are oil-soluble (or fat-soluble) and may be encapsulated within low melting point temperature materials.

The sunscreen agents useful according to the disclosure may be employed in an amount of from about 0.1% to about 40% by weight, such as from about 0.5% to about 30% by weight, such as from about 1% to about 25% by weight, based on the total weight of the solid wax particle comprising the aqueous dispersion.

The pigments/dyes/colorants of the wax dispersion and/or cosmetic composition according to various embodiments of the present disclosure may optionally be soluble in oil and include, but are not limited to, permanent, semi-permanent and/or temporary dyes.

Representative and non-limiting pigments include white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium.

The direct dyes and oxidation dyes which may be used according to the disclosure are those dyes employed to color hair and textile fabrics. Representative oxidation dyes include, but are not limited to para-phenylenediamines, bis(phenyl)alkylenediamines, para-aminophenols, ortho-aminophenols and heterocyclic bases, and the addition salts thereof. Representative direct dyes include, but are not limited to, azo, methane, carbonyl, azine, nitro (hetero)aryl, tri(hetero)arylmethane, porphyrin, phthalocyanin direct dyes, and natural direct dyes.

The wax dispersion according to various embodiments of the present disclosure may further comprise sub-micron-sized to micron-sized particles of silica, talc, and/or clays, which include, but are not limited to, montmorillonite, bentonite, hectorite, attapulgite, sepiolite, laponite, smectite, kaolin, and their mixtures.

These clays can be modified with a chemical compound chosen from quaternary ammoniums, tertiary amines, amine acetates, imidazo lines, amine soaps, fatty sulphates, alkylarylsulphonates, amine oxides and their mixtures.

Mention may be made, as organophilic clays, of quaternium-18 bentonites, such as those sold under the names Bentone 3, Bentone 38 or Bentone 38V by Rheox, Tixogel VP by United Catalyst or Claytone 34, Claytone 40 or Claytone XL by Southern Clay; stearalkonium bentonites, such as those sold under the names Bentone 27 by Rheox, Tixogel LG by United Catalyst or Claytone AF or Claytone APA by Southern Clay; or quaternium-1 8/benzalkonium bentonites, such as those so Id under the names Claytone HT or Claytone PS by Southern Clay.

Suitable silicas may include pyrogenic silicas obtained by high temperature hydrolysis of a volatile silicon compound in an oxyhydrogen flame, producing a finely divided silica. This process makes it possible in particular to obtain hydrophilic silicas which exhibit a large number of silanol groups at their surfaces.

It is possible to chemically modify the surface of the silica by a chemical reaction for the purpose of decreasing the number of silanol groups. It is possible in particular to substitute silanol groups by hydrophobic groups: a hydrophobic silica is then obtained.

The hydrophobic groups can be:
trimethylsiloxyl groups, which are obtained in particular by treatment of pyrogenic silica in the presence of hexamethyldisilazane. Silicas thus treated are also named “Silica silylate.”
dimethylsilyloxyl or polydimethylsiloxane groups, which are obtained in particular by treatment of pyrogenic silica in the presence of polydimethylsiloxane or of dimethyldichlorosilane. Silicas thus treated are also named “Silica dimethyl silylate.”

The pyrogenic silica may exhibit a particle size that is sub-micron sized or micron sized, for example ranging from about 5 to 200 nm.

The silica, talc, and/or clays may be present in an amount of from about 0.01% to about 10% by weight, such as from about 0.5% to about 2% by weight, based on the weight of the aqueous dispersion.

The wax dispersion according to various embodiments of the present disclosure may further comprise perfumes or fragrances to aid in the fragrance of the product and provide a time-release effect. The perfume can have a dual effect by not only providing a pleasant fragrance but also to provide shine to a treated substrate. The perfumes may be present in an amount of from about 0.01% to about 10% by weight, such as from about 0.5% to about 2% by weight, based on the weight of the aqueous dispersion.

Methods of Making the Wax Dispersion

The wax dispersion may, according to at least certain exemplary embodiments, be obtained by means of a process comprising at least the following steps (the Wax Dispersion Protocol), although any process which produces a wax dispersion as described herein is contemplated to be within the scope of the disclosure:

emulsifying a mixture containing at least one wax having a melting point or melting temperature greater than 35° C., a surfactant mixture comprising a nonionic surfactant and an ionic surfactant, and water at an emulsification temperature above the melting point of the at least one wax. If two or more waxes are used, the emulsification temperature should be higher than the melting point of the wax with the higher or highest melting point,

subjecting the mixture to a process leading to the production of solid wax particles, at a temperature at least 5 to 10° C. above the emulsification temperature of the mixture used in the preceding step, and

cooling the dispersion thus obtained.

It is pointed out that the combination of ingredients in the first step of the process and the execution of the second step with heating may be cumulative conditions for obtaining the solid wax particles according to the disclosure in a controlled manner, resulting in solid wax particles that are calibrated to certain properties (e.g., melting point, size, and shape), if desired. Thus, the nature of the process exerted on the wax-surfactant-water mixture can determine the properties of the particles to be obtained.

The process may, optionally, also include a step of diluting the continuous phase of the mixture before the cooling step.

As used herein, the expression “process leading to the production of solid wax particles” is intended to denote an action of shear type. This shearing action can, for example, be accomplished by mixing the wax-surfactant-water mixture using a homogenizer/mixer at a specified speed.

By way of example, by using different speeds of mixing, different particle sizes can be achieved such as those ranging from 0.5-100 microns, 1-50 microns, 2-25 microns, 8-20 microns, 2-10 microns, and even less than 1 micron. For example, particle sizes of up to about 20 microns, such as up to about 15 microns, up to about 12.5 microns, up to about 10 microns, up to about 7.5 microns, up to about 5 microns, or up to about 2.5 microns may be obtained. In at least one embodiment, particles having a size of about 10 microns are formed. It is also possible to use other shearing processes such as those described and referred to in US2006/0292095 and US2006/0263438.

The emulsification temperature may, in various embodiments, be greater than 40° C. and/or less than 95° C.

In accordance with exemplary embodiments employing the process described above, the solid wax particles may be obtained as a dispersion in an aqueous and/or water-soluble continuous phase. Such a dispersion may also be described as an oil-in-water emulsion or an oil-in-water dispersion.

The solid wax particles in the wax dispersion may, in at least some embodiments, not aggregate in the dispersion in which they are obtained, and their granulometric specificities in terms of size and distribution index may be conserved therein.

The aqueous and/or water-soluble continuous phase that is suitable for use according to the disclosure may comprise water or a combination of water and a water-soluble solvent.

Among the water-soluble solvents that may be used in the wax dispersions in accordance with various embodiments according to the disclosure, mention may be made especially of lower monoalcohols containing from 1 to 5 carbon atoms, such as ethanol and isopropanol, glycols, glycol ethers, and polyols, for instance glycerol, ethylene glycol, propylene glycol, butylene glycol, caprylyl glycol, hexylene glycol, dipropylene glycol, diethylene glycol, xylitol, sorbitol, mannitol, maltitol, and polyethylene glycol or mixtures thereof, C3 and C4 ketones, and C2-C4 aldehydes and mixtures thereof.

For the purposes of the present disclosure, the term “water-soluble solvent” is intended to denote a compound that is liquid at room temperature and water-miscible (miscibility in water of greater than 50 percent by weight at 25° C. and at atmospheric pressure).

According to yet another exemplary embodiment, the wax dispersions may comprise demineralized or deionized water as the continuous aqueous phase.

Compositions

The compositions containing the at least one film former and at least one wax dispersion further comprise components that are typically found in such compositions, e.g. cosmetic compositions and/or personal care products.

As used herein, the expression “compositions, e.g. cosmetic compositions and/or personal care products” is intended to include compositions, whether considered cosmetic compositions, personal care products, etc., irrespective of their common referent, that are intended for use in making up, caring for, etc., keratinous substrates, including but not limited to the hair, skin, lips, and nails.

By way of non-limiting example, the compositions typically comprise a carrier and at least one auxiliary component.

Carrier

The compositions according to various embodiments comprise a carrier which includes, but is not limited to water, volatile and non-volatile organic solvents, silicones, polyols, glycols, glycol ethers, oils, and mixtures thereof.

When the organic solvent is a volatile solvent, the amount of the volatile organic solvent generally ranges from greater than 0 (e.g., about 0.01%) to about 99%, and in some embodiments from greater than 0 to about 55%, and in some embodiments from greater than 0 to about 2%, by weight, based on the total weight of the composition. In certain embodiments, the amount of volatile organic solvent does not exceed 55%.

In certain embodiments, the carrier is a cosmetically, dermatologically or physiologically acceptable carrier that is non toxic, wherein the compositions can be applied onto keratinous substrates such the skin, lips, hair, scalp, lashes, brows, nails or any other cutaneous region of the body. The cosmetically, dermatologically or physiologically acceptable carrier may comprise water and/or one or more of the organic solvents, silicones, polyols, glycols, glycol ethers, oils.

The carrier can be employed in an amount of from about 20% to about 99% by weight, or such as from about 40% to about 95% by weight, about 60% to about 90%, or such as from about 80% to about 85% by weight, based on the total weight of the composition. In one exemplary embodiment, the carrier may be about 25% to about 75% by weight, such as about 35% to about 65%, or about 45% to about 55% by weight, based on the total weight of the composition.

Auxiliary Components

The compositions comprising the aqueous dispersion of the present disclosure may additionally contain one or more additional or auxiliary components chosen from, for example, solvents, preservatives, fragrances, thickeners or texturizers, liquid lipids/oils, rheology modifiers, sunscreen agents, pigments/colorants/dyes, silica, clays, humectants and moisturizing agents, emulsifying agents, structuring agents, propellants, surfactants, shine agents, conditioning agents, cosmetically, dermatologically and pharmaceutically active agents, vitamins, plant extracts, and additional film-formers and coalescents/plasticizers. One of skill in the art will be able to select appropriate types and amounts of additional cosmetic ingredients, based on, for example, the type of cosmetic composition being formulated and the desired properties thereof. By way of example only, any of the additional components set forth above for the wax dispersion may be included in the compositions. In certain exemplary embodiments, such auxiliary components may be present in the compositions according to the disclosure in a combined amount ranging from about 10% to about 80%, such as about 15% to about 60%, about 25% to about 40%, or about 30% to about 35%.

Representative liquid lipids comprise oils, triglycerides and liquid fatty substances such as mineral oil, avocado oil, camellia oil, turtle oil, macadamia nut oil, corn oil, mink oil, olive oil, rape seed oil, egg yolk oil, sesame oil, persic oil, wheat germ oil, sasanqua oil, castor oil, linseed oil, safflower oil, cottonseed oil, perilla oil, soybean oil, peanut oil, tea seed oil, kaya oil, rice bran oil, chinese-wood oil, japanese-wood oil, jojoba oil, germ oil, triglycerol, glyceryl trioctanoate, pentaerythritol tetraoctanote, and glyceryl triisopalmitate.

Representative rheology modifiers include, but are not limited to, thickening agents, and gelling agents. Broadly, the rheology modifier(s) that may be useful in the practice of embodiments of the disclosure include those conventionally used in cosmetics such as polymers of natural origin and synthetic polymers.

Representative rheology-modifiers that may be used in the practice of embodiments according to the disclosure may be chosen from nonionic, anionic, cationic, and amphoteric polymers, including acrylate-based polymers, polysaccharides, polyamino compounds, amphiphilic polymers, and other rheology modifiers such as cellulose-based thickeners (e.g., hydroxyethylcellulose, hydroxypropylcellulose, carboxymethylcellulose, cationic cellulose ether derivatives, quaternized cellulose derivatives, etc.), guar gum and its derivatives (e.g., hydroxypropyl guar, cationic guar derivatives, etc.), gums such as gums of microbial origin (e.g., xanthan gum, scleroglucan gum, etc.), and gums derived from plant exudates (e.g., gum arabic, ghatti gum, karaya gum, gum tragacanth, carrageenan gum, agar gum and carob gum), pectins, alginates, and starches, crosslinked homopolymers of acrylic acid or of acrylamidopropane-sulfonic acid, associative polymers, non-associative thickening polymers, and water-soluble thickening polymers.

The surfactants that may be employed as auxiliary agents may be chosen from anionic, cationic, nonionic and amphoteric surfactants such as those described above.

The shine agents may be chosen from silicones, alkoxylated silicones, oils, ethoxylated oils, fats, esters, transesters, hydrocarbons, quats and mixtures thereof.

Non-limiting examples of shine agents include Amodimethicone, Dimethicone, Dimethiconol, Cyclemethicone, Phenyltrimethicone, Aminopropyl Phenyltrimethicone, Trimethyl Pentaphenyl Trisiloxane, Cetyl Dimethicone, Alkyl Dimethicone, Potassium Dimethicone PEG-7 Pantheyl Phosphate, Olive oil, Jojoba oil, Apricot oil, Avocado oil, Castor oil, Lanolin, Squalane, Capric/Caprylic Triglyceride, Octyl Palmitate, Isopropyl Palmitate, Isopropyl Myristate, Mineral oil, Petrolatum, Polyquaternium-4, Polyquaternium-11, Behentrimonium Methosulfate, Benetrimonium Chloride and mixtures thereof.

The compositions according to various embodiments of the present disclosure may additionally comprise one or more additives chosen from pearlescent agents, opacifying agents, fragrances, sequestering agents, softeners, antifoams, wetting agents, spreading agents, dispersants, plasticizers, mineral fillers, colloidal minerals, peptizers, preserving agents, and pH adjusters.

The compositions of the present disclosure may be in the form of an aqueous system, e.g. an aqueous dispersion, a simple or complex emulsion (oil-in-water (o/w), water-in-oil (w/o), silicone-in-water and/or water-in-silicone emulsion types) such as a cream or a milk, in the form of a gel or a cream-gel, or in the form of a lotion, a powder or a solid tube, and any other form that is convenient to the consumer.

The compositions of the present disclosure may be applied onto keratinous substrates, such as skin, lips, nails, and hair.

Cosmetic compositions and/or personal care products according to various exemplary embodiments of the disclosure may have improved and/or increased ease of removability, relative to similar compositions not having the at least one wax dispersion as described herein. In various embodiments, ease of removability relates to ease of removing the composition from the keratinous substrate with warm (e.g. about 50° C. or higher) water. Optionally, conventional cleansing agents such as soap or make-up remover may also be used.

As such, the disclosure also relates to methods of improving or increasing the ease of removability in compositions having at least one film former, such as at least one latex film former, by incorporating wax dispersions as described herein into said cosmetic compositions and/or personal care products.

As used herein, the expression “at least one” means one or more and thus includes individual components as well as mixtures/combinations.

The terms “a” and “the” as used herein are understood to encompass the plural as well as the singular.

The term “comprising” (and its grammatical variations) as used herein is used in the inclusive sense of “having” or “including” and not in the exclusive sense of “consisting only of”.

Other than in the operating examples, or where otherwise indicated, all numbers expressing quantities of ingredients and/or reaction conditions are to be understood as being modified in all instances by the term “about.” As used herein, the modifier “about” means within the limits that one of skill in the art would expect with regard to the particular quantity defined; this may be, for example, in various embodiments, +10% of the indicated number, +5% of the indicated number, +2% of the indicated number, +1% of the indicated number, +0.5% of the indicated number, or +0.1% of the indicated number.

“Keratinous substrates” (and its grammatical variations) as used herein, include, but are not limited to skin, lips, and nails, and keratinous fibers such as hair and eyelashes.

“Substituted” as used herein, means comprising at least one substituent. Non-limiting examples of substituents include atoms, such as oxygen atoms and nitrogen atoms, as well as functional groups, such as acyloxyalky groups, carboxylic acid groups, amine or amino groups, acylamino groups, amide groups, halogen containing groups, ester groups, thiol groups, sulphonate groups, thiosulphate groups, siloxane groups, and polysiloxane groups. The substituent(s) may be further substituted.

As used herein, the phrases “salts thereof” and “derivatives thereof” are intended to mean all salts and derivatives comprising the same functional structure as the compound they are referring to, and that have similar properties.

As used herein, the term “applying a composition” onto a substrate and variations of this phrase are intended to mean contacting the substrate, for example, a keratinous substrate such as skin or hair, with at least one of the compositions according to embodiments of the disclosure, in any manner.

As used herein, “formed from,” means obtained from chemical reaction of, wherein “chemical reaction,” includes spontaneous chemical reactions and induced chemical reactions. As used herein, the phrase “formed from,” is open ended and does not limit the components of the composition to those listed.

The term “treat” (and its grammatical variations) as used herein refers to the application of the aqueous dispersion and compositions containing the dispersion onto the surface of a substrate.

The compositions and methods according to the present disclosure can comprise, consist of, or consist essentially of the elements and limitations described herein, as well as any additional or optional ingredients, components, or limitations described herein or otherwise known and useful in the art.

The following examples of dispersions and compositions are intended to illustrate exemplary embodiments according to the disclosure, without limiting the scope as a result. The percentages are given on a weight basis.

### EXAMPLES

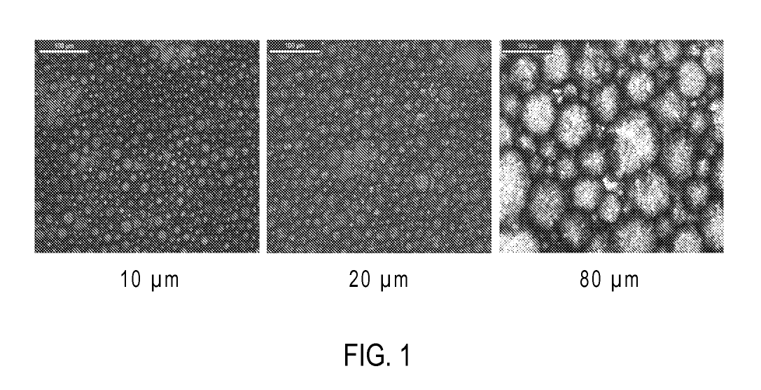

Three wax dispersions having particle sizes of 10 microns, 20 microns, and 80 microns were prepared using beeswax, according to the Wax Dispersion Protocol set forth above. FIG. 1 shows micrographs of each of these beeswax dispersions, via optical microscopy.

Next, the following seven exemplary extended-wear mascara compositions were prepared, as follows:

| Component | Amount A | Amount B | Amount C | Amount D | Amount E | Amount F | Amount G |
|---|---|---|---|---|---|---|---|
| Joncryl 77 | 18.93 | 18.93 | 18.93 | 18.93 | 18.93 | 18.93 | 18.93 |
| Dermacryl | 19.14 | 19.14 | 19.14 | 19.14 | 19.14 | 19.14 | 19.14 |
| AQF | | | | | | | |
| Tributyl | 1.48 | 1.48 | 1.48 | 1.48 | 1.48 | 1.48 | 1.48 |
| Citrate | | | | | | | |
| Pigment Paste | 12.72 | 12.72 | 12.72 | 12.72 | 12.72 | 12.72 | 12.72 |
| Beeswax/ | 0 | 5/10μ | 5/20μ | 5/80μ | 10/10μ | 10/20μ | 10/80μ |
| particle size | | | | | | | |
| Water | QS | QS | QS | QS | QS | QS | QS |

The mascara compositions were individually prepared by combining all the materials into one container and mixed for 3 minutes at 2750 rpm using the Speedmixer DAC 400.1 FVZ (FlackTek Inc.) at room temperature. The final mascara compositions were transferred to the mascara components.

The mascara compositions were then applied on fake lashes for 30 strokes, and the smudge and water resistances were evaluated after immersing the fake eyelash into artificial sebum and water for 24 hrs. After 24 hrs, the fake eyelashes were tested for smudge and water resistance by brushing them on to water color paper 10 times at a rate of 12 rpm. After 10 strokes of the fake eyelashes on to water color paper, the paper was air dried and examined for the level of transfer from the various mascara formula.

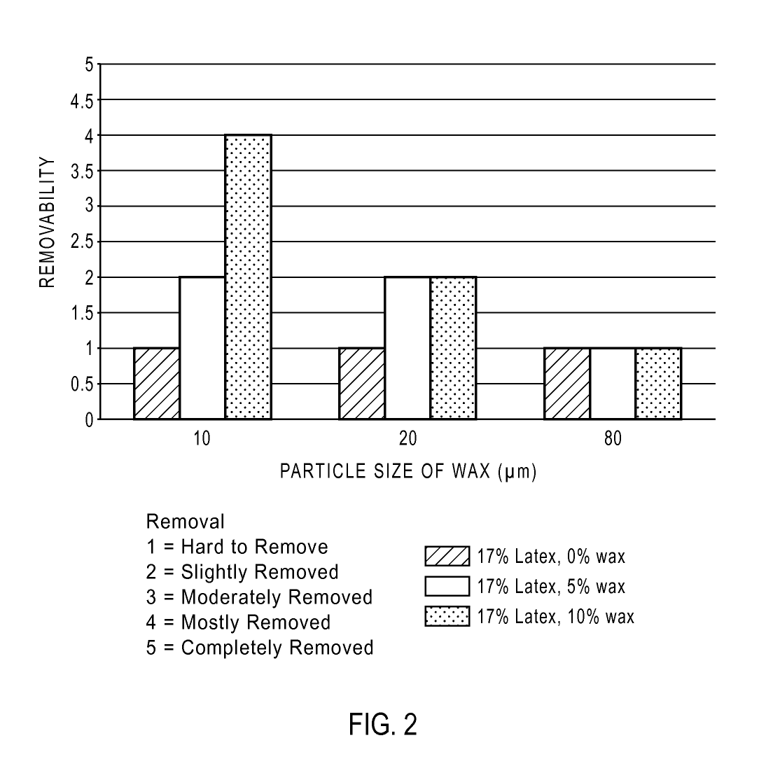

The ease of removability of each of the seven compositions was also evaluated, and the results are seen in FIG. 2.

It is to be understood that the foregoing describes exemplary embodiments of the disclosure only, and that modifications may be made therein without departing from the spirit or scope of the disclosure.

1. A mascara composition comprising:
(a) at least one latex film former; and
(b) a wax dispersion comprising
(i) solid wax particles having a particle size ranging from equal to or greater than about 1 micron up to about 60 microns;
(ii) a surfactant mixture comprising:
a. at least one nonionic surfactant; and
b. at least one ionic surfactant; and
(iii) water.

2. The mascara composition of claim 1, wherein the at least one solid wax particle comprises particles having a particle size ranging up to about 20 microns.

3. The mascara composition of claim 1, wherein the at least one solid wax particle comprises particles having a particle size ranging up to about 10 microns.

4. The mascara composition of claim 1, wherein the least one wax is chosen from beeswax, hydrogenated myristyl olive esters, hydrogenated stearyl olive esters, VP/eicosene copolymer, ditrimethyloylpropane tetrastearate, and silsesquioxane resin wax.

5. The mascara composition of claim 1, wherein the solid wax particles are present in an amount of from about 20% to about 45% by weight, based on the total weight of the wax dispersion.

6. The mascara composition of claim 1, wherein the solid wax particles are present in an amount of from about 30% to about 40% by weight, based on the total weight of the wax dispersion.

7. The mascara composition of claim 1, wherein the at least one latex film former is present in an amount of at least about 10% by weight of the mascara composition.

8. The mascara composition of claim 1, wherein the at least one latex film former is present in an amount of at least about 15% by weight of the mascara composition.

9. The mascara composition of claim 1, wherein the at least one latex film former is present in an amount of at least about 20% by weight of the mascara composition.

10. The mascara composition of claim 1, wherein the at least one nonionic surfactant has an HLB of at least 5 and is chosen from polyethylene glycol ethers of glyceryl esters, sorbitan esters, and mixtures thereof.

11. The mascara composition of claim 1, wherein the at least one nonionic surfactant is chosen from PEG-30 glyceryl stearate and sorbitan palmitate.

12. The mascara composition of claim 1, wherein the ionic surfactant comprises at least one cationic surfactant.

13. The mascara composition of claim 1, wherein the ionic surfactant comprises at least one cationic surfactant chosen from cetrimonium chloride, behentrimonium chloride, dipalmitoylethyl hydroxyethylmonium methosulfate, distearoylethyl hydroxyethyl-monium methosulfate, and mixtures thereof.

14. The mascara composition of claim 1, wherein the ionic surfactant comprises at least one anionic surfactant.

15. The mascara composition of claim 1, wherein the ionic surfactant comprises at least one anionic surfactant chosen from acyl glutamates, alkyl sulfates and their salts, alkyl ether sulfates and their salts, acyl glutamates, alkyl ether carboxylates, and mixtures thereof.

16. The mascara composition of claim 1, wherein the ionic surfactant comprises at least one anionic surfactant chosen from disodium stearoyl glutamate and sodium stearoyl glutamate.

17. The mascara composition of claim 1, wherein the at least one ionic surfactant is present in an amount of from about 5% to about 30% by weight, based on the total weight of the surfactant mixture.

18. The mascara composition of claim 1, wherein the at least one ionic surfactant is present in an amount of from about 5% to about 20% by weight, based on the total weight of the surfactant mixture.

19. The mascara composition of claim 1, wherein the surfactant mixture is present in an amount of from about 1% to about 5% by weight, based on the total weight of the wax dispersion.

20. A method of increasing the removability of a composition comprising at least one latex film former, said method comprising incorporating a wax dispersion into the composition, wherein said wax dispersion comprises:
(a) solid wax particles having a particle size ranging from equal to or greater than about 1 micron up to about 60 microns;
(b) a surfactant mixture comprising:
(i) at least one nonionic surfactant; and
(ii) at least one ionic surfactant; and
(c) water,
wherein the at least one ionic surfactant is present in an amount ranging from about 5% to about 30% by weight, based on the total weight of the surfactant mixture.

21. The method of claim 20, wherein the at least one solid wax particle comprises particles having a particle size ranging up to about 20 microns.

22. The method of claim 20, wherein the at least one solid wax particle comprises particles having a particle size ranging up to about 10 microns.

23. The method of claim 20, wherein the least one wax is chosen from beeswax, hydrogenated myristyl olive esters, hydrogenated stearyl olive esters, VP/eicosene copolymer, ditrimethyloylpropane tetrastearate, and silsesquioxane resin wax.

24. The method of claim 20, wherein the solid wax particles are present in an amount of from about 20% to about 45% by weight, based on the total weight of the wax dispersion.

25. The method of claim 20, wherein the solid wax particles are present in an amount of from about 30% to about 40% by weight, based on the total weight of the wax dispersion.

26. The method of claim 20, wherein the at least one latex film former is present in an amount of at least about 10% by weight of the composition.

27. The method of claim 20, wherein the at least one latex film former is present in an amount of at least about 15% by weight of the composition.

28. The method of claim 20, wherein the at least one latex film former is present in an amount of at least about 20% by weight of the composition.

29. The method of claim 20, wherein the at least one nonionic surfactant has an HLB of at least 5 and is chosen from polyethylene glycol ethers of glyceryl esters, sorbitan esters, and mixtures thereof.

30. The method of claim 20, wherein the at least one nonionic surfactant is chosen from PEG-30 glyceryl stearate and sorbitan palmitate.

31. The method of claim 20, wherein the ionic surfactant comprises at least one cationic surfactant.

32. The method of claim 20, wherein the ionic surfactant comprises at least one cationic surfactant chosen from cetrimonium chloride, behentrimonium chloride, dipalmitoylethyl hydroxyethylmonium methosulfate, distearoylethyl hydroxyethyl-monium methosulfate, and mixtures thereof.

33. The method of claim 20, wherein the ionic surfactant comprises at least one anionic surfactant.

34. The method of claim 20, wherein the ionic surfactant comprises at least one anionic surfactant chosen from acyl glutamates, alkyl sulfates and their salts, alkyl ether sulfates and their salts, acyl glutamates, alkyl ether carboxylates, and mixtures thereof.

35. The method of claim 20, wherein the ionic surfactant comprises at least one anionic surfactant chosen from disodium stearoyl glutamate and sodium stearoyl glutamate.

36. (canceled)

37. The method of claim 20, wherein the at least one ionic surfactant is present in an amount of from about 5% to about 20% by weight, based on the total weight of the surfactant mixture.

38. The method of claim 20, wherein the surfactant mixture is present in an amount of from about 1% to about 5% by weight, based on the total weight of the wax dispersion.
